(19) United States
(12) Patent Application Publication
(10) Pub. No.: US 2022/0018629 A1
(43) Pub. Date: Jan. 20, 2022

(54) FIREARM BARREL NUT AND HANDGUARD MOUNTING PLATE ASSEMBLY

(71) Applicant: WEV Works, LLC, Grand Forks, ND (US)

(72) Inventor: Jordan Senff, Grand Forks, ND (US)

(21) Appl. No.: 17/337,089

(22) Filed: Jun. 2, 2021

Related U.S. Application Data
(60) Provisional application No. 63/034,089, filed on Jun. 3, 2020.

(51) Int. Cl. F41C 23/16; F41A 21/48; F41C 23/18

(52) U.S. Cl. CPC F41C 23/16; F41C 23/18; F41A 21/482

(57) ABSTRACT

A firearm barrel nut and handguard mounting assembly configured to addresses alignment issues and over torquing of the barrel nut relative to an upper receiver, as well as enabling ease in removal and replacement of a handguard. The firearm barrel nut and handguard mounting assembly including a barrel nut defining a channel positioned between a proximal end and a distal end, and one or more handguard mounting plates, each handguard mounting plate including a handguard retention portion and a barrel nut connection portion, the barrel nut connection portion configured to mate with the channel of the barrel nut to create an interference fit between the barrel nut and the one or more handguard mounting plates, thereby inhibiting movement of the one or more handguard mounting plates relative to the barrel nut.

### RELATED APPLICATION

The present application claims the benefit of U.S. Provisional Application No. 63/034,089 filed Jun. 3, 2020, which is hereby incorporated herein in its entirety by reference.

### TECHNICAL FIELD

The present disclosure relates generally to firearms, and more particularly to a barrel nut and handguard mounting mechanism for use in firearms including the AR-10, AK-15, M-16, and variants thereof.

### BACKGROUND

The ArmaLite AR-10 was developed by Eugene Stoner in the late 1950s as a lightweight assault rifle for military use. The basic AR-10 design had a direct impingement, rotating bolt configured to accept 7.62×51 mm NATO (.308 Winchester) cartridges. In 1957, the AR-10 design was rescaled and substantially modified by ArmaLite to accommodate 5.56×45 mm NATO (.223 Remington) cartridges, and given the designation AR-15. ArmaLite sold its patent rights to the AR-10 and AR-15 to Colt Firearms in 1959. After subsequent modifications, the AR-15 was adopted by the US military as the M-16 rifle. With the expiration of the ArmaLite patents in 1977, other manufacturers began producing their own variants of the firearm, commonly known as AR-15 style rifles.

Today, the AR-15 rifle has become one of the most beloved rifles in the United States, and has been referred to as “America's rifle” by the National Rifle Association (NRA). One innovative feature of the AR-15 rifle is its distinctive two-part upper and lower receiver and its modular design enabling the ease in the interchangeability and replacement of parts. As civilian ownership of AR-15 style rifles grew, numerous manufacturers began producing “improved” aftermarket modules, assemblies, or parts with features not found on factory rifles. Due to the AR-15 rifle's modular construction, individuals with an average mechanical aptitude can substitute these aftermarket parts with the original factory parts to customize their rifle. Due to the vast assortment of aftermarket parts and accessories currently on the market, the AR-15 style rifle has been referred to as the “Swiss Army knife of rifles.”

A few of the interchangeable components or parts of the AR-15 rifle include the barrel and handguard. In most variants, the barrel is connected to the upper receiver via a barrel nut. When attaching the barrel to the upper receiver, the barrel nut is slid along the barrel to a desired position. A barrel extension portion of the barrel is then inserted within the upper receiver, and the barrel nut is tightened to hold the barrel in a desired position relative to the upper receiver.

To hold a proximal end of the handguard in place, typically a handguard snap ring, weld spring and delta ring are positioned over the barrel extension prior to insertion into the upper receiver. Most barrel nuts are generally cylindrically shaped devices having a series of outer spokes which circumvolve an outer surface of the barrel nut and define between them a plurality of troughs, which are configured to allow passage of a gas tube therethrough.

In the installation of a handguard, which are typically divided into a top portion and a bottom portion, a distal end of the handguard is positioned within a handguard, while the user grips the delta ring to compress the weld spring to expose the barrel nut, thereby enabling the proximal portion of the handguard to be positioned over the barrel nut. As the handguard typically includes an inner set of knobs configured to correspond to the plurality of troughs of the barrel nut, the handguard must be aligned with the barrel nut for proper positioning. The delta ring can then be released, thereby holding the handguard in position relative to the barrel under a natural bias of the weld spring.

In practice, removal and replacement of a handguard from an AR-15 variant firearm can be a tedious and time-consuming practice. The present disclosure addresses this concern.

### SUMMARY OF THE DISCLOSURE

Embodiments of the present disclosure provide a firearm barrel nut and handguard mounting assembly configured to addresses alignment issues and over torquing of the barrel nut relative to an upper receiver, as well as enabling ease in removal and replacement of a handguard.

One embodiment of the present disclosure provides firearm barrel nut and handguard mounting assembly, including the barrel nut and one or more handguard mounting plates. The barrel nut can include a tubular wall extending between a proximal end and a distal end. An interior portion of the tubular wall in proximity to the proximal end can define a threaded portion configured to be operably coupled to a corresponding threaded portion of an upper receiver. An exterior portion of the tubular wall can define a hexagonal shape in proximity to the distal end. The exterior portion of the tubular wall can further define a channel positioned between the proximal end and the distal end. Each of the one or more handguard mounting plates can include a handguard retention portion and a barrel nut connection portion. The handguard retention portion can include structure defining one or more apertures shaped and sized to enable a fastener to pass therethrough for operably coupling to a handguard. The barrel nut connection portion configured to mate with the channel of the barrel nut to create an interference fit between the barrel nut and the one or more handguard mounting plates, thereby inhibiting movement of the one or more handguard mounting plates relative to the barrel nut.

Another embodiment of the present disclosure provides a firearm including a receiver, a barrel, a barrel nut configured to securely fasten the barrel to the receiver, the barrel nut comprising a tubular wall extending between a proximal end and a distal end, an interior portion of the tubular wall in proximity to the proximal end defining a threaded portion configured to be operably coupled to a corresponding threaded portion of an upper receiver, an exterior portion of the tubular wall defining a polygonal shape in proximity to the distal end, the exterior portion of the tubular wall further defining a channel positioned between the proximal end and the distal end, a handguard, and one or more handguard mounting plates configured to operably couple the handguard to the barrel, each handguard mounting plate comprising a handguard retention portion and a barrel nut connection portion, the handguard retention portion including structure defining one or more apertures shaped and sized to enable a fastener to pass therethrough for operably coupling to a handguard, the barrel nut connection portion configured to mate with the channel of the barrel nut to create an interference fit between the barrel nut and the one or more handguard mounting plates, thereby inhibiting movement of the one or more handguard mounting plates relative to the barrel nut.

In one embodiment, at least one of the barrel nut or one or more handguard mounting plates are constructed of a magnesium alloy. In one embodiment, the channel defined by the barrel nut is in the form of a V-shaped groove. In one embodiment, the barrel nut connection portion is in the form of a V-shaped protuberance, configured for mating receipt within the V-shaped groove of the channel defined by the barrel nut. In one embodiment, the channel defined by the barrel nut has at least one of a square, rectangular, polygonal, semi-circular or semi-elliptical cross-section. In one embodiment, the barrel nut connection portion defines a protuberance having at least one of a square, rectangular, polygonal, semi-circular or semi-elliptical cross-section. In one embodiment, the proximal end of the tubular wall has a larger outer diameter than a distal end of the tubular wall. In one embodiment, the tubular wall of the barrel nut further defines at least one transition, representing at least one of a step, ramp or other change in a diameter of the exterior portion of the tubular wall. In one embodiment, each handguard mounting plate defines at least two apertures. In one embodiment, the one or more apertures defined by each handguard mounting plate includes a chamfer to enable flush mounting of a fastener against an exterior surface of the handguard retention portion.

The summary above is not intended to describe each illustrated embodiment or every implementation of the present disclosure. The figures and the detailed description that follow more particularly exemplify these embodiments.

### BRIEF DESCRIPTION OF THE DRAWINGS

The disclosure can be more completely understood in consideration of the following detailed description of various embodiments of the disclosure, in connection with the accompanying drawings, in which:

FIG. 1 is an exploded, perspective view depicting a firearm upper receiver, barrel, barrel nut, and handguard of the prior art.

FIG. 2A is an exploded, perspective view of a barrel nut and one or more handguard mounting plate, in accordance with an embodiment of the disclosure.

FIG. 2B depicts a perspective view of the barrel nut and one or more handguard mounting plate of FIG. 2A, assembled with a handguard, in accordance with an embodiment of the disclosure.

FIGS. 3A-C depicts various views of a barrel nut, in accordance with an embodiment of the disclosure.

FIG. 4A-E depicts various views of a handguard mounting plate, in accordance with an embodiment of the disclosure.

While embodiments of the disclosure are amenable to various modifications and alternative forms, specifics thereof shown by way of example in the drawings will be described in detail. It should be understood, however, that the intention is nut to limit the disclosure to the particular embodiments described. On the contrary, the intention is to cover all modifications, equivalents, and alternatives falling within the spirit and scope of the subject matter as defined by the claims.

### DETAILED DESCRIPTION

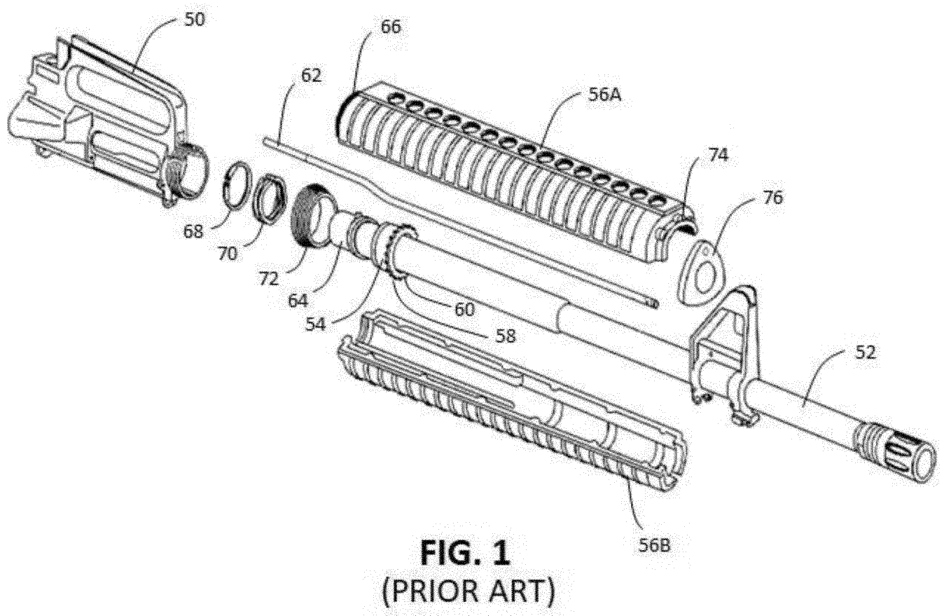

Referring to FIG. 1, an exploded, perspective view of a conventional upper receiver 50, barrel 52, barrel nut 54, and handguard 56A/B of the prior art is depicted. Most barrel nuts 54 are generally cylindrically shaped devices having a series of inner threads, which are configured to attach to a portion of the upper receiver 50. The inner portion of the barrel nut 54 is configured to both receive and hold the barrel 52, as well as to attach the barrel 52 to the upper receiver 50 of the firearm. As depicted, the barrel nut 54 defines a series of outer spokes 58 which circumvolve an outer surface of the barrel nut 54. These spokes 58 define between them a plurality of troughs 60, which are configured to allow passage of a gas tube 62 therethrough.

The upper receiver 50 includes a series of outer threads configured to interconnect with the complementary configured threads of the barrel nut 54. When attaching the barrel 52 to the upper receiver 50, the barrel nut 54 is slid along the barrel 52 to a desired position. A barrel extension portion 64 of the barrel 52 is then inserted within the upper receiver 50, and the barrel nut 54 is tightened to hold the barrel 52 in a desired position relative to the upper receiver 50.

The handguard 56 is divided into a top portion 56A and a bottom portion 56B. To hold a proximal end 66 of the handguard 56 in place, typically a handguard snap ring 68, weld spring 70 and delta ring 72 are positioned over the barrel extension 64 prior to insertion into the upper receiver 50. In order to operably couple the handguard 56 to the barrel 52, a distal end 74 of the handguard 56 is positioned within a handguard cap 76 positioned on the barrel 52. Thereafter, the delta ring 72 is pushed towards the upper receiver 50 to compress the weld spring 70, thereby exposing the barrel nut 54 to enable a proximal portion 66 of the handguard 56 to be positioned over the barrel nut 54. As the handguard typically includes an inner set of knobs configured to correspond to the plurality of troughs 60 of the barrel nut 54, the handguard 56 must be aligned with the barrel nut 54 for proper positioning. The delta ring 72 can then be released, thereby securing the handguard 56 in position relative to the barrel 52 under a natural bias of the weld spring 70.

With most existing models of the prior art, removal and replacement of the handguard 56 is a tedious process that requires a significant amount of time, as the user must continually compress the weld spring 70 with one hand, while simultaneously manipulating the top and bottom portions of the handguard 56A/B for proper alignment relative to the barrel nut 54. Removal of the barrel 52 from the upper receiver 50 requires the use of a specialized tool that fits over the spokes 58 of the barrel nut 54. Further, as one trough 60 of the barrel nut 54 must be aligned with a top of the upper receiver 50 to allow passage of the gas tube 62 therethrough, proper rotational alignment of the barrel nut 54 at the prescribes torque can be difficult. In many cases, users over torque the barrel nut threads to ensure proper alignment, which can result in the connection between the barrel 52 and the upper receiver 50 being compromised or functioning improperly. In addition, cross threading of the barrel nut 54 and/or upper receiver 50 can occur.

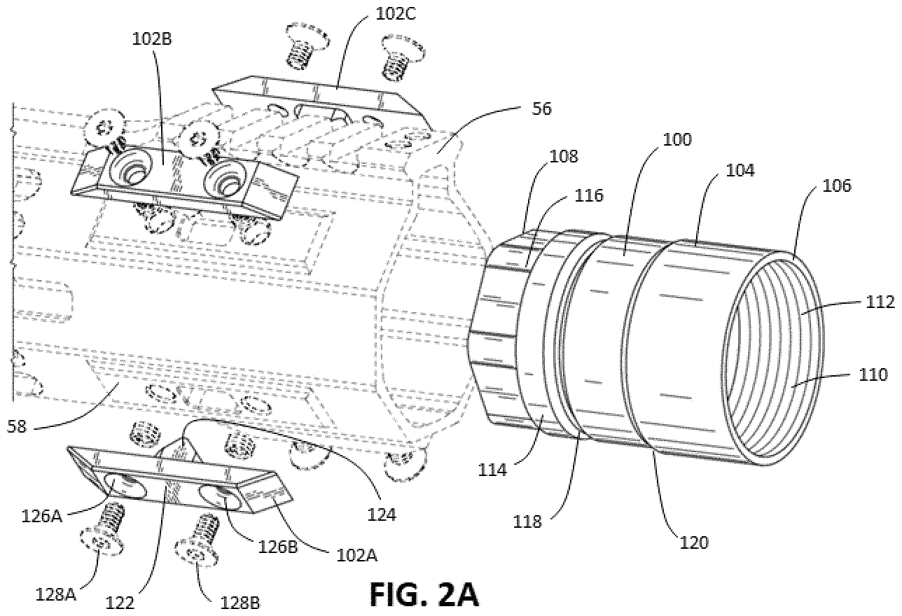

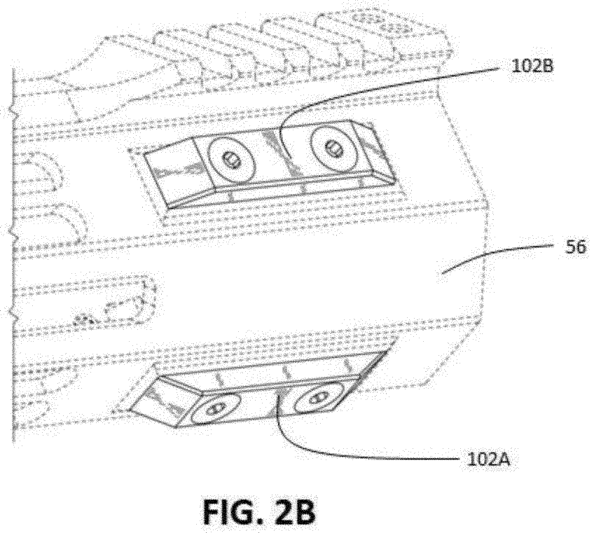

The present disclosure provides an improved barrel nut 100 and handguard mounting plate assembly 102, which addresses alignment issues and over torquing of the barrel nut relative to the upper receiver, as well as enabling ease in removal and replacement of the handguard. Referring to FIG. 2A, an exploded, perspective view of a barrel nut 100, handguard 56, and one or more handguard mounting plates 102A-C, is depicted in accordance with an embodiment of the disclosure. FIG. 2B depicts a perspective view of the barrel nut 100, handguard 56 and handguard mounting plates 102 of FIG. 2A as assembled.

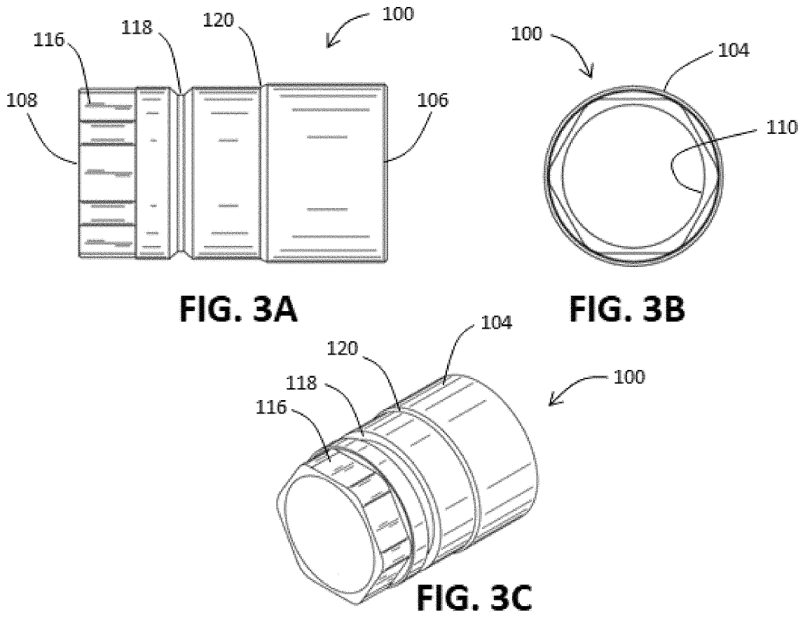

With additional reference to FIGS. 3A-C, the barrel nut 100 can generally be a cylindrically shaped sleeve having a tubular wall 104 extending between proximal end 106 and a distal end 108. An interior portion or surface 110 of the tubular wall 104 in proximity to the proximal end 106 can define one or more threads 112 configured to operably coupled to a corresponding threaded portion of an upper receiver 50. An exterior portion or surface 114 of the tubular wall 104 in proximity to the distal end 108 can define a generally polygonal (e.g., hexagon, square, etc.) shaped surface 116, which can be sized according to US or metric standards for compatibility with one or more tools to tighten, loosen and otherwise manipulate the barrel nut 100 relative to the upper receiver 50.

In some embodiments, an exterior surface of the tubular wall 104 can further define a channel, groove or other detent 118 generally located between the proximal end 106 and the distal end 108. For example, as depicted in FIG. 3A, in some embodiments, the channel 118 can be in the form of a V-shaped groove; although other channel 118 configurations are also contemplated. For example, in some embodiments, the channel 118 can have a square, rectangular, polygonal, semi-circular, semi-elliptical, or other shaped cross-section. Further, in some embodiments, the barrel nut 100 can include at least one transition 120, representing a step, ramp or other change in a diameter of the exterior surface 114 of the barrel nut 100, for example where a proximal end portion 106 of the tubular wall 104 may have a larger outer diameter than a distal end portion 108 of the tubular wall 104.

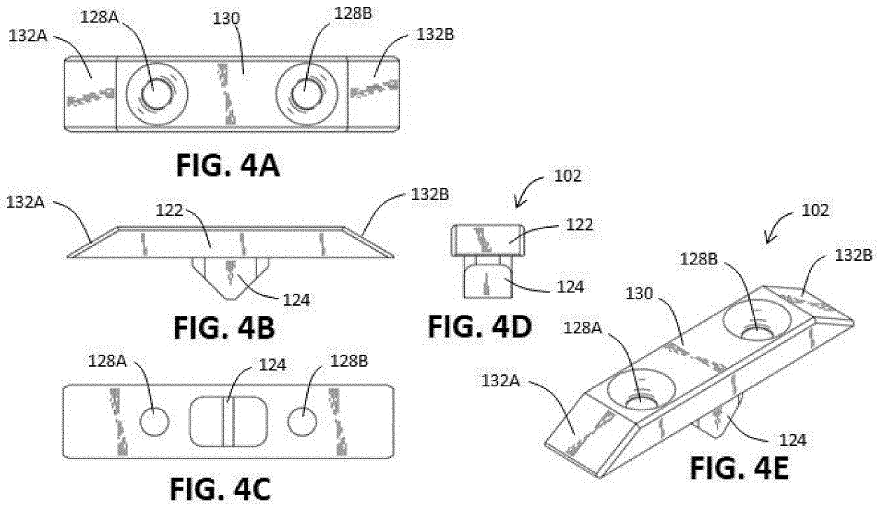

With additional reference to FIGS. 4A-E, each handguard mounting plate 102 can include a handguard retention portion 122 and a barrel nut connection portion 124. In some embodiments, the handguard retention portion 122 can include structure defining one or more apertures 126A/B, through which a fastener 128A/B can traverse, thereby enabling the handguard retention portion 122 to be operably coupled to the exterior of a handguard 56. For example, in one embodiment, the handguard mounting plate 102 can include two apertures 126A/B, although the use of a greater or lesser number of apertures is also contemplated. In some embodiments, the one or more apertures 126A/B can be chamfered to enable flush mounting of the fasteners 128A/B against an exterior surface 130 of the handguard retention portion 122. The exterior surface 130 of the handguard retention portion 122 can be configured in a variety of shapes and sizes. For example, as depicted in FIGS. 2A-B & 4A-E, in one embodiment, there exterior surface 130 can include one or more beveled surfaces 132 configured to present a low-profile when mated with the handguard 56, thereby presenting a clean appearance. Other configurations of the handguard retention portion 122 are also contemplated.

In some embodiments, the barrel nut connection portion 124 can be configured as a wedge, or other shape compatible with the channel, groove or other detent 118 defined on the exterior of the barrel nut 100. For example, as depicted in FIGS. 2A-B & 4A-E, in some embodiments, the barrel nut connection portion 124 can be in the form of a V-shaped protuberance; although other barrel nut connection portion 124 configurations are also contemplated. Accordingly, in some embodiments, a tightening of the fastener 128A/B can be configured to both cause the handguard retention portion 122 to be securely fastened to the handguard 56, as well as to forcibly urge the barrel nut connection portion 124 into surface engaging contact with the channel, groove or other detent 118 of the barrel nut 100. In some embodiments, the force imparted by the barrel nut connection portion 124 upon the barrel nut 100 can be sufficiently strong enough to create an interference fit between the barrel nut 100 and the one or more handguard mounting plate assemblies 102, thereby inhibiting movement (e.g., rotation, translation, etc.) of the handguard 56 relative to the barrel nut 100.

In some embodiments, the handguard 56 can be constructed of a tubular member, as opposed to a traditional two-part handguard, such as that depicted in FIG. 1. Further, as depicted in FIG. 2A, in some embodiments, the handguard 56 can define one or more recesses or cutouts 58 into which a portion of the handguard mounting plate 102 can reside, thereby creating a lower profile, smoother surface of the handguard 56. In some embodiments, at least one of the barrel nut 100, handguard mounting plates 102 and/or handguard 56 can be constructed of a magnesium alloy; although the use of other materials in the construction of the barrel nut 100, handguard mounting plates 102, and/or handguard 56 is also contemplated.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when nut described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are nut incorporated by reference herein unless expressly included herein.

For purposes of interpreting the claims, it is expressly intended that the provisions of 35 U.S.C. § 112(f) are nut to be invoked unless the specific terms “means for” or “step for” are recited in a claim.

What is claimed is:

1. A firearm barrel nut and handguard mounting assembly, comprising:
a barrel nut including a tubular wall extending between a proximal end and a distal end, an interior portion of the tubular wall in proximity to the proximal end defining a threaded portion configured to be operably coupled to a corresponding threaded portion of an upper receiver, an exterior portion of the tubular wall defining a polygonal shape in proximity to the distal end, the exterior portion of the tubular wall further defining a channel positioned between the proximal end and the distal end; and
one or more handguard mounting plates, each handguard mounting plate including a handguard retention portion and a barrel nut connection portion, the handguard retention portion including structure defining one or more apertures shaped and sized to enable a fastener to pass therethrough for operably coupling to a handguard, the barrel nut connection portion configured to mate with the channel of the barrel nut to create an interference fit between the barrel nut and the one or more handguard mounting plates, thereby inhibiting movement of the one or more handguard mounting plates relative to the barrel nut.

2. The assembly of claim 1, wherein at least one of the barrel nut or one or more handguard mounting plates are constructed of a magnesium alloy.

3. The assembly of claim 1, wherein the channel defined by the barrel nut is in the form of a V-shaped groove.

4. The assembly of claim 3, wherein the barrel nut connection portion is in the form of a V-shaped protuberance, configured for mating receipt within the V-shaped groove of the channel defined by the barrel nut.

5. The assembly of claim 1, wherein the channel defined by the barrel nut has at least one of a square, rectangular, polygonal, semi-circular or semi-elliptical cross-section.

6. The assembly of claim 5, wherein the barrel nut connection portion defines a protuberance having at least one of a square, rectangular, polygonal, semi-circular or semi-elliptical cross-section.

7. The assembly of claim 1, wherein the proximal end of the tubular wall has a larger outer diameter than a distal end of the tubular wall.

8. The assembly of claim 1, wherein the tubular wall of the barrel nut further defines at least one transition, representing at least one of a step, ramp or other change in a diameter of the exterior portion of the tubular wall.

9. The assembly of claim 1, wherein each handguard mounting plate defines at least two apertures.

10. The assembly of claim 1, wherein the one or more apertures defined by each handguard mounting plate includes a chamfer to enable flush mounting of a fastener against an exterior surface of the handguard retention portion.

11. A firearm comprising:
a receiver;
a barrel;
a barrel nut configured to securely fasten the barrel to the receiver, the barrel nut comprising a tubular wall extending between a proximal end and a distal end, an interior portion of the tubular wall in proximity to the proximal end defining a threaded portion configured to be operably coupled to a corresponding threaded portion of an upper receiver, an exterior portion of the tubular wall defining a polygonal shape in proximity to the distal end, the exterior portion of the tubular wall further defining a channel positioned between the proximal end and the distal end;
a handguard; and
one or more handguard mounting plates configured to operably couple the handguard to the barrel, each handguard mounting plate comprising a handguard retention portion and a barrel nut connection portion, the handguard retention portion including structure defining one or more apertures shaped and sized to enable a fastener to pass therethrough for operably coupling to a handguard, the barrel nut connection portion configured to mate with the channel of the barrel nut to create an interference fit between the barrel nut and the one or more handguard mounting plates, thereby inhibiting movement of the one or more handguard mounting plates relative to the barrel nut.

12. The firearm of claim 11, wherein at least one of the barrel nut or one or more handguard mounting plates are constructed of a magnesium alloy.

13. The firearm of claim 11, wherein the channel defined by the barrel nut is in the form of a V-shaped groove.

14. The firearm of claim 13, wherein the barrel nut connection portion is in the form of a V-shaped protuberance, configured for mating receipt within the V-shaped groove of the channel defined by the barrel nut.

15. The firearm of claim 11, wherein the channel defined by the barrel nut has at least one of a square, rectangular, polygonal, semi-circular or semi-elliptical cross-section.

16. The firearm of claim 15, wherein the barrel nut connection portion defines a protuberance having at least one of a square, rectangular, polygonal, semi-circular or semi-elliptical cross-section.

17. The firearm of claim 11, wherein the proximal end of the tubular wall has a larger outer diameter than a distal end of the tubular wall.

18. The firearm of claim 11, wherein the tubular wall of the barrel nut further defines at least one transition, representing at least one of a step, ramp or other change in a diameter of the exterior portion of the tubular wall.

19. The firearm of claim 11, wherein each handguard mounting plate defines at least two apertures.

20. The firearm of claim 11, wherein the one or more apertures defined by each handguard mounting plate includes a chamfer to enable flush mounting of a fastener against an exterior surface of the handguard retention portion.
